(19) United States
(12) Patent Application Publication
(10) Pub. No.: US 2004/0235012 A1
(43) Pub. Date: Nov. 25, 2004

(54) Method of detecting antibiotic resistance in microorganisms

(76) Inventors: Rainer Hammann, Wiesenbach (DE); Alain Rambach, Paris (FR); Susan Kircher, Reistertown, MD (US); Nancy Dick, Upperco, MD (US)

Correspondence Address: PATTON BOGGS LLP, 8484 WESTPARK DRIVE, SUITE 900, MCLEAN, VA, 22102, US

(21) Appl. No.: 10/753,417

(22) Filed: Jan. 9, 2004

Related U.S. Application Data
(60) Provisional application No. 60/438,992, filed on Jan. 10, 2003.

(51) Int. Cl. C12Q 1/68

(52) U.S. Cl. 435/6

(57) ABSTRACT

The current invention relates to a method of detecting antibiotic resistant microorganisms, comprising culturing microorganisms in or on a chromogenic agar that has been supplemented with at least one β-lactam antibiotic, provided that the β-lactam is not oxacillin. The current invention also relates to kits used for the detection of antibiotic resistant organisms.

### BACKGROUND OF THE INVENTION

1. Field of the Invention

The current invention relates to a method of detecting antibiotic resistant microorganisms, comprising culturing microorganisms in or on a chromogenic agar that has been supplemented with at least one β-lactam antibiotic, provided that the β-lactam is not oxacillin.

2. Background of the Invention

Staphylococcus aureus (S. aureus) is one of the most frequently isolated human pathogens. The frequency of methicillin resistance found in S. aureus has increased dramatically in hospitals and nursing facilities worldwide. In addition, methicillin resistant S. aureus (MRSA) are also emerging in the community setting. In recent years low-level resistant strains, (also referred to as borderline resistant strains) have been responsible for infections in Japan, Europe, and the United States. These borderline resistant strains are extremely heterogeneous in their resistance levels and are characterized by having minimal inhibitory concentration (MIC) breakpoints at or near the susceptibility breakpoint. These borderline resistant strains may be misdiagnosed as susceptible strains, using currently recommended non-molecular based methods. (Felten et al., 2002. Evaluation of Three Techniques for Detection of Low-Level Methicillin-Resistant Staphylococcus aureus (MRSA): a Disk Diffusion Method with Cefoxitin and Moxalactam, the Vitek 2 System, and the MRSA-Screen Latex Agglutination Test. JCM. 40: 2766-2771; Swenson, J. M. et al., 2001. Performance of Eight Methods, Including Two New Rapid Methods, for Detection of Oxacillin Resistance in a Challenge Set of Staphylococcus aureus Organisms. J. Clin. Microbiol. 39:3785-3788; Gerberding, J. L. et al., 1991. Comparison of conventional susceptibility tests with direct detection of penicillin-binding protein 2a in borderline oxacillin-resistant strains of Staphylococcus aureus. Antimicrob. Agents Chemother. 35: 2574-2579; Resende, C. A., A. M. Figueiredo. 1997. Discrimination of methicillin-resistant Staphylococcus aureus from borderline-resistant and susceptible isolates by different methods. J. Med. Microbiol. 46: 145-149; Dickinson, T. M., G. L. Archer. 2000. Phenotypic Expression of Oxacillin Resistance in Staphylococcus aureus.: Roles of mecA Transcriptional Regulation and Resistant-Subpopulation Selection. Antimicrob. Agents Chemother. 44: 1616-1623; BBL Quality Control and Product Information Manual for Plated Media. Oxacillin Screen Agar) Thus, accurate detection and identification of all forms of MRSA is paramount to appropriately isolate and treat the organism, and to control its spread.

Molecular methods, e.g., PCR and DNA hybridization, used to detect methicillin resistance also require a pure isolate, and few laboratories offer such costly procedures. While molecular methods offer quicker results than standardized susceptibility tests (same day vs. 24 hour), the molecular-based methods, which are based on detection of gene sequences, cannot differentiate between functional and nonfunctional genes. For example, S. sciuri possess a native mecA gene (responsible for encoding penicillin binding proteins (PBPs)), which may appear similar to MRSA on DNA hybridization. (Murray, P. R. et al., 1999. Manual of Clinical Microbiology, 7^{th} edition. ASM pg 276) Additionally, there is not a current molecular method for detection of MRSA directly from specimens.

The National Committee for Clinical Laboratory Standards (NCCLS) recommends the use of oxacillin in a disc diffusion method, MIC or oxacillin agar screen for the detection of MRSA. (NCCLS. 2000. Performance standards for Dilution Antimicrobial Susceptibility Tests for Bacteria that grow aerobically. Approved standard, 5^{th} ed. Document M7-A5. NCCLS, Wayne, Pa.; NCCLS. 2000 Performance Standard for Antimicrobial Disk Susceptibility Tests; Approved Standard-7^{th} ed. Document M2-A7. NCCLS, Wayne, Pa.) The disc diffusion, MIC and agar screening methods require a pure isolate, which usually takes 18-24 hours to prepare, and an additional 24 hours to perform the detection test, resulting in a total of 48 hours for detection of MRSA. The agar screen method uses Mueller Hinton agar (a non-selective and non-differential base) containing oxacillin, which is inoculated with a standardized inoculum of a purified culture. The disc diffusion and MIC methods also require a standardized inoculum. All of the detection procedures discussed are satisfactory for the confirmation high-level resistant strains; however, these methods may not detect low-level resistant strains, because the oxacillin MIC of these strains is at or just above the susceptible breakpoint (oxacillin 4-8ug/ml). Additionally, the agar screen method cannot be used directly on a clinical sample.

Mannitol Salt Agar, with oxacillin, as an agar screen or as a disc diffusion method has been recommended for screening isolates. (Kampf, G. et al., 1998. Evaluation of Mannitol Salt Agar for Detection of Oxacillin Resistance in Staphylococcus aureus by Disk Diffusion and Agar Screening. J. Clin. Microbiol. 36: 2254-2257) The mannitol salt agar with oxacillin was initially described as a primary isolation medium for MRSA in 1992. (Enk, R. A. et al., 1992. Use of primary isolation medium for recovery of methicillin-resistant Staphylococcus aureus. J. Clin. Microbiol. 30: 504-505) Recently, Oxoid (UK) developed a commercial media, Oxacillin Resistance Screening Agar Base (ORSAB™), which is a selective medium that presumptively offers identification of MRSA from specimens. ORSAB™ contains a Mannitol Salt agar base with aniline blue for detection of mannitol fermentation and a combination of polymixin B and oxacillin to enable detection of MRSA strains from specimens. Several studies have been published on ORSAB™, comparing it to current methods, and the ORSAB™ method demonstrated good sensitivity towards MRSA, but appears to lack specificity with clinical specimens. Additionally, non-MRSA strains may grow on ORSAB™, necessitating additional testing to rule out MRSA when used as a primary screen media. (Simor, A. F. et al., 2001. Evaluation of a New Medium, Oxacillin Resistance Screening Agar Base, for the Detection of Methicillin-Resistant Staphylococcus aureus from Clinical Specimens. J. Clin. Microbiol. 39: 3422-3422)

The shortcomings of oxacillin, the most widely used antibiotic for MRSA detection, are well documented. For example, inoculum size, growth conditions, such as, NaCl concentration, incubation time and temperature and pH are important in accurately detecting the presence of MRSA, when using oxacillin as a screening compound. Tight control and close monitoring of all of the aforementioned growth conditions are critical when using an oxacillin based media, because slower growing resistant populations will not be detected. (Swenson, J. M. et al., 2001. Optimal Inoculation Methods and Quality Control for the NCCLS Oxacillin Screen Test for Detection of Oxacillin Resistance in Staphylococcus aureus. J. Clin. Microbiol. 39: 3781-3784; Chambers, H. F. 1988. Methicillin Resistant Staphylococci. Clin. Micro. Reviews. 1: 173-186; de Lencastre, H. et al., 1991. Multiple mechanisms of methicillin resistance and improved methods for detection in clinical isolates of Staphylococcus aureus. Antimicrob. Agents Chemother. 35: 632-639; Tomasz, A. et al., 1991. Stable classes of phenotypic expression in methicillin-resistant clinical isolates of staphylococci. Antimicrob. Agents Chemother. 35:124-129) Additionally, the effectiveness of using oxacillin to detect methicillin resistant microorganisms is diminished when a low concentration of inoculum is used. Indeed, most MRSA are heterogeneous in their expression of resistance, where only one in 10⁴ to 10⁸ cells exhibit reduced sensitivity to methicillin. As a result of this heterogeneous expression, the concentration of the inoculum is critical for accurate detection, when using oxacillin as a primary screening agent. Thus, the current methods, which use oxacillin as a screening compound, may provide inaccurate results (both false negatives and false positives), whether or not the proper growth conditions are maintained. Accordingly, there is a continuing need for a non-molecular based method of accurately screening and detecting MRSA.

### SUMMARY OF THE INVENTION

The current invention relates to a method of detecting antibiotic resistant microorganisms, comprising culturing microorganisms in or on a chromogenic agar that has been supplemented with at least one β-lactam antibiotic, provided that the β-lactam is not oxacillin. The current invention also relates to kits used for the detection of antibiotic resistant organisms.

### BRIEF DESCRIPTION OF THE DRAWINGS

N/A

### DETAILED DESCRIPTION OF THE INVENTION

The current invention relates to a method of detecting antibiotic resistant microorganisms, comprising culturing microorganisms in or on a chromogenic agar that has been supplemented with at least one β-lactam antibiotic, provided that the β-lactam is not oxacillin. As used herein, the terms “detecting” or “detection” are used to mean the identification or isolation of a microorganism, which distinguishes or separates the microorganism of interest from other microorganisms on a phenotypic or genetic basis. The detection can be a quantitative difference or a qualitative difference. The detection method can be, for example, by color, using the specific chromogenic agents. Furthermore, the difference between the two or more distinguished microorganisms can be minimal, in that as long as a genetic or phenotypic difference is highlighted by performing the methods of the invention, detection has occurred.

As used herein, a chromogenic agar medium is an agar containing a chromogenic agent, wherein the cultured microorganisms that survive will produce a detectable color in or on the medium, indicating the microorganisms' viability under the current culture conditions. In one embodiment, the invention relates to a method of detecting antibiotic resistant microorganisms, comprising culturing microorganisms in or on a chromogenic agar medium for S. aureus, characterized in that it contains at least one chromogenic agent. The chromogenic agar medium is also characterized in that it has been supplemented with at least one β-lactam antibiotic, wherein the β-lactam is not oxacillin. Examples of chromogenic agents include, but are not limited to, 5-bromo-6-chloro-3-indoxyl phosphate, 5-bromo-4-chloro-3-indoxyl galactoside “X-gal”), 5-bromo-4-chloro-3-indoxyl glucuronide (“X-glucuronide”) and/or 5-bromo-4-chloro-3-indoxyl glucoside (“X-glu”).

As used herein, the term antibiotic is used as one of ordinary skill in the art would recognize the term. In one embodiment, the antibiotic to which the microorganism is resistant is a β-lactam. As used here, “β-lactam” is a compound that contains the characteristic β-lactam ring, as is recognized in the art. The presently known β-lactams include the penicillin-type compounds, cephalosporins, carbepenems, monobactams and β-lactamase inhibitors. As used herein, a “penicillin-type compound” is a compound containing the characteristic penicillin structure, which, of course, includes the β-lactam ring and a five-membered thiazolidine ring structure. Examples of penicillin-type compounds include, but are not limited to, penicillin G, penicillin V, methicillin, oxacillin, cloxacillin, dicloxacillin, nafcillin, ampicillin, amoxicillin, carbenicillin, ticarcillin, azlocillin, mezlocillin, piperacillin and amdinocillin, or any salt or anhydrous thereof.

Other β-lactams include cephalosporins, which, as used herein, is a compound containing the characteristic cephalosporin structure, which includes the β-lactam ring attached to a six-membered dihydrothiazine ring. Examples of cephalosporins include, but are not limited to, any first, second, third and fourth generation cephalosporin, including, but not limited to, cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome or any salt or anhydrous thereof.

Additional β-lactams include the carbapenems, monobactams and β-lactamase inhibitors. As used herein, a carbapenem is a compound containing the characteristic carbapenem structure, which includes the β-lactam ring fused to a five-membered ring, as highlighted in The Manual of Clinical Microbiology, 6^{th} Ed., Murray et al eds. ASM Press, pp 1281-1286. Examples of carbapenems include, but are not limited to, imipenem and meropenem, or any salt or anhydrous thereof. As used herein, the monobactams are a monocyclic β-lactam compound that do not have a ring structure attached to the β-lactam ring. An example of a monobactam includes, but is not limited to aztreonam, or any salt or anhydrous thereof. Finally, β-lactamase inhibitors are also included in the β-lactams. β-lactam inhibitors also contain the β-lactam ring and examples of β-lactamase inhibitors include, but are not limited to clavulanic acid, sulbactam and tazobactam or any salt or anhydrous thereof.

The structures of the various classes of the β-lactam compounds can be found in The Manual of Clinical Microbiology, 6^{th} Ed., Murray et al. ASM Press, Washington D.C., pp1281-1286, which is herein incorporated by reference.

As used herein the term “antibiotic resistant” is used to mean that at least some portion of a population of microorganism can survive and/or actively grow and divide in the presence of the antibiotic. For example, antibiotic resistance is used to mean that the bacteria does not lyse or is not otherwise destroyed by the antibiotic. Antibiotic resistance can also mean that the microorganism actively grows and divides in the presence of the antibiotic.

Preferably, the microorganism to be assayed is from the genus Staphylococcus, as is defined in Bergey's Manual of Systemic Bacteriology, 4^{th} Ed., which is herein incorporated by reference. Some of the defining characteristics of the Staphylococcus genus include, but are not limited to, the organism being (a) Gram-positive, (b) cluster-forming, (c) nonmotile, (d) non-spore-forming, (e) facultative anaerobe, (f) fermentation of glucose produces mainly lactic acid, (g) catalase-positive, (h) coagulase-positive, (i) golden yellow colony on agar. Furthermore, Staphylococcus bacteria are also characterized by being part of the normal flora of humans found in nasal passages, skin and mucous membranes. Additionally, Staphylococcus are also common pathogens of humans, and can cause a wide range of adverse conditions including, but not limited to, suppurative infections, food poisoning and toxic shock syndrome. Most preferably, the microorganism is Staphylococcus aureus.

As used herein, the term “administration” is used to mean any means of delivering the screening agent to the microorganism. The administration of the screening agent can be directly onto cultured cells, for example, or the agent can be directly inoculated into liquid growth media, and can be at any concentration effective to screen the microorganisms. The administration can also be in the form of supplementing agar, such as a chromogenic agar, such that an agar plate, when used to culture microorganisms, contains the screening agent, at any concentration. The concentration of screening agent used will depend on a variety of factors, including, but not limited to, the exact compound used, the route of administration, the growth conditions in which the bacteria are being kept, which include such factors as salt concentration, temperature, concentration of oxygen and carbon dioxide, inoculum concentration and the type of growth media used. In one particular embodiment, the concentration of screening agent used is from about 0.01 μg/ml to about 100 μg/ml. More particularly, the concentration of the screening agent is from about 0.1 μg/ml to about 50 μg/ml. Even more particularly, the range of concentration is from about 1 μg/ml to about 40 μg/ml. In another embodiment, the inoculum size is from about 10 CFU/ml to about 10⁸ (CFU/ml). Particularly, the inoculum is from about 10³ CFU/ml to about 10⁵ CFU/ml. As used herein, the term “screening agent” is use to mean the specific compound (β-lactam) that is administered to the microorganism to determine whether it is resistant to a particular antibiotic.

The methods of the current invention are performed to determine whether a microorganism is resistant to penicillin-type compounds. The phrase “methicillin resistant” is used herein as it is used in that art; namely that the microorganisms are resistance to all β-lactam compounds, not just the specific methicillin compound. In one particular embodiment, the antibiotic towards which the microorganism is resistant is the specific compound methicillin.

In another embodiment, the β-lactam that is administered to the microorganism to determine its antibiotic resistance is a cephalosporin. In particular, the cephalosporin can be any one or a combination of cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome. In one particular embodiment, the cephalosporin which is administered to the microorganism is cefoxitin.

Cephalosporins, especially cefoxitin, are superior to oxacillin for the detection of heterogeneous populations of MRSA and those expressing low level methicillin resistance. This is most likely attributed to an enhanced ability of the cephalosporins to induce PBP2a production, without the lethality of oxacillin or methicillin.

Fewer limitations of the cephalosporin containing media also offer advantages. Additional NaCl and incubation at no higher than 35° C. are required for oxacillin containing media, whereas additional NaCl and temperature limitations are not significant factors when culturing with cephalosporins. For example, the incubation temperature used in the current methods range from about 27° C. to about 42° C. In particular, the incubation temperature is from about 33 to 37° C. Although, supplementation with additional NaCl, up to a concentration of about 5%, produced optimal results, concentrations as low as 2.5% NaCl also performed well. Concentrations as low as 0.01% may also provide satisfactory results.

The invention also relates to kits for detecting the presence of antibiotic resistant microorganisms, comprising at least one β-lactam, provided that the β-lactam is not oxacillin. In particular, the kit comprises a cephalosporin. More particularly, the kit comprises a cephalosporin which is selected from the group consisting of cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome. Ever more preferable, the kit comprises cefoxitin.

### EXAMPLES

### EXAMPLES 1

### Preparation of Cefoxitin-Containing Agar

To prepare a solution of cefoxitin at a concentration of 6 ug/ml, in DI water, prepare a stock solution of (0.2 g/25 ml water). The stock was diluted to the final concentration by using 750 ul stock solution/ liter.

The cefoxitin working solution was added aseptically to a final concentration of 6 ug/ml to CHROMagar™ Staph aureus base (see below). The final concentrations of the various ingredients of the agar are listed in table 1, below.

TABLE 1

| | g/L |
|---|---|
| CALCIUM CHLORIDE 2H2O | 0.1840 |
| GE90M | 30.0000 |
| YEAST EXTRACT | 10.0000 |
| NaCl | 40.0000 |
| COLISTIN SULFATE | 0.0100 |
| NALIDIXIC ACID, SODIUM SALT | 0.0030 |
| Tween 80 | 2.0000 |
| SETEXAM AGAR | 14.0000 |
| X-GLU | 0.0500 |
| X-GAL | 0.0500 |
| X-GLUCORONIDE | 0.0500 |
| MAG PHOS | 0.1 |
| DEFEROX | 0.05 |
| TRIZMA | 0.1 |
| CEFOXITIN | 0.006 |
| FUNGIZONE | 0.003 |

First, the raw materials, according to the chromogenic media formulation, were added to DI water, and heated to boiling for 1 minute. After boiling, the media was autoclaved at about 121° C. for 15 minutes and subsequently cooled to 45-50° C. Additional raw materials, magenta phosphate, deferoxamine, trizma, and amphotericin B, as well as the cefoxitin solution were added aseptically to the media. The complete media was then dispensed into 100 mm petri dishes and allowed to solidify on a level surface.

### EXAMPLE 2

### Culturing of S. aureus on Cefoxitin-Agar Plates

Cultures of difficult to detect MRSA and borderline S. aureus strains were obtained from clinical sites, and the ATCC (ATCC strains were used for internal QC), and prepared. The culture was allowed to grow for 2 hours. A sample of culture was added to 0.5 McFarland turbidity in Trypticase Soy Broth (TSB) to estimate the concentration of bacteria as measured by CFU (colony forming units). Next, the cultures were diluted to about 10⁵ CFU, using sterile DI water. Finally, the chromogenic plates, containing the cefoxitin were streaked with a loop. The cultures were then incubated at 35° C. to 37° C. for 24 hours.

Results of screening tests, comparing screening with oxacillin, safoxitin, moxalactam, cefmetazole and ceftazidime, are presented in Tables 2 and 3.

TABLE 2

| | OX - | FOX - | MOX - | CMZ - | CAZ - |
|---|---|---|---|---|---|
| | 4 | 6 | 16 | 2.25 | 16 |
| Sensitivity | 5.9 | 88.2 | 88.2 | 100 | 33.3 |
| Specificity | 100 | 100 | 100 | 50 | 100 |
| Positive Predictive Value | 100 | 100 | 100 | 90 | 100 |
| Negative Predictive Value | 23.8 | 71.4 | 71.4 | 100 | 20 |
| Accuracy | 27.3 | 90.9 | 90.9 | 90.9 | 42.9 |
| Total Number of Strains | 22 | 22 | 22 | 11 | 21 |
| Evaluated | | | | | |

Table Legend: All values are expressed in percentage.
OX - 4: oxacillin at 4 ug/ml (working solution);
FOX - 6: safoxitin at 6 ug/ml (working solution);
MOX - 16: moxalactam at 16 ug/ml (working solution);
CMZ - 2.25: cefmetazole at 2.25 ug/ml (working solution);
CAZ - 16: ceftazidime at 16 ug/ml (working solution)

TABLE 3

| | | | E-Test |
|---|---|---|---|
| | | | MIC |
| Strains Tested | mecA | PBP′2 | (ug/ml) |
| T4031 | + | + | 6 |
| T4033 | + | + | 1.5 |
| T2130 | + | + | 32 |
| T4035 | + | + | 8 |
| T2110 | + | + | 8 |
| T775 | + | + | 8 |
| T2109 | + | + | 32 |
| 5597 | + | + | 32 |
| 3425 | − | − | 0.38 |
| 5596 | + | + | 0.5 |
| 5595 | + | + | 0.5 |
| 34 | + | + | 16 |
| 3424 | − | − | 1.5 |
| 5599 | + | + | 16 |
| 5594 | + | + | 0.38 |
| 5598 | + | + | 32 |
| 2325 | − | − | 0.19 |
| 31 | − | − | 0.25 |
| ATCC 43300 | | + | |
| ATCC 33592 | | + | |
| ATCC 25923 | − | | |
| 3421 | + | | >4 |

Table Legend: mecA: presence (+) or absence (−) of the mecA gene; PBP2: presence (+) or absence (−) of the PBP2 protein; E-Test ™ is manufactured by AD Biodisk and the E-Test was performed according to the manufacturers suggested protocol; MIC is minimum inhibitory concentration of oxacillin used in the E-Test

All references cited herein are hereby incorporated by reference.

What is claimed is:

1. A method of detecting antibiotic resistant microorganisms, comprising administering at least one β-lactam antibiotic to said microorganisms, wherein said β-lactam antibiotic is not oxacillin.

2. The method of claim 1, wherein said administration comprises culturing said microorganisms in or on a chromogenic agar medium for S. aureus, said chromogenic agar medium comprising at least one chromogenic agent selected from the group consisting of 5-bromo-6-chloro-3-indoxyl phosphate and 5-bromo-4-chloro-3-indoxyl glucoside, said chromogenic agar further comprising at least one β-lactam antibiotic, wherein said β-lactam is not oxacillin.

3. The method of claim 2, wherein said microorganisms belong to the Staphylococcus genus.

4. The method of claim 3, wherein said microorganisms are species Staphylococcus aureus (S. aureus).

5. The method of claim 4, wherein said S. aureus is resistant to penicillin-type compounds.

6. The method of claim 5, wherein said penicillin-type compound is methicillin.

7. The method of claim 6, wherein said β-lactam is a cephalosporin.

8. The method of claim 7, wherein said cephalosporin is selected from the group consisting of cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome.

9. The method of claim 8, wherein said cephalosporin is cefoxitin.

10. The method of claim 1, wherein said β-lactam is a cephalosporin.

11. The method of claim 10, wherein said cephalosporin is selected from the group consisting of cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome.

12. The method of claim 11, wherein said cephalosporin is cefoxitin.

13. A kit for detecting the presence of antibiotic resistant microorganisms comprising at least one β-lactam, wherein said at least one β-lactam is not oxacillin.

14. The kit of claim 13, wherein said wherein said β-lactam is a cephalosporin.

15. The kit of claim 14, wherein said cephalosporin is selected from the group consisting of cephalothin, cefazolin, cephradine, cephalexin, cefadroxil, cefamandole, cefoxitin, cefaclor, cefuroxime, cefuroxime axetil, loracarbef, cefonicid, cefotetan, ceforanide, cefotaxime, cefpodoxime proxetil, ceftizoxime, cefiximeceftriaxone, cefoperazone, ceftazidime, moxalactam, cefipime and cefpirome.

16. The kit of claim 15, wherein said cephalosporin is cefoxitin.
